(19) United States
(12) Patent Application Publication
(10) Pub. No.: US 2017/0014449 A1
(43) Pub. Date: Jan. 19, 2017

(54) SITE-SPECIFIC EPIGENETIC EDITING

(71) Applicant: Elwha LLC, Bellevue, WA (US)

(72) Inventors: Mahalaxmi Gita Bangera, Renton, WA (US); Michael H. Baym, Cambridge, MA (US); Roderick A. Hyde, Redmond, WA (US); Wayne R. Kindsvogel, Seattle, WA (US); Gary L. McKnight, Bothell, WA (US); Elizabeth A. Sweeney, Seattle, WA (US)

(73) Assignee: Elwha LLC, a limited liability company of the State of Delaware

(21) Appl. No.: 14/797,283

(22) Filed: Jul. 13, 2015

(51) Int. Cl. A61K 35/12; C12N 15/90; C12N 15/85

(52) U.S. Cl. CPC A61K 35/12; C12N 15/85; C12N 15/907

(57) ABSTRACT

Disclosed herein include various embodiments related to site-specific epigenetic editing of biological cells. Various embodiments relate to fusion protein constructs and methods of using the same for site-specific epigenetic editing of biological cells. Various embodiments relate to fusion protein constructs that utilize endogenous epigenetic editing effector agents for site-specific editing of biological cells.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a partial view of an embodiment described herein relating to genomic DNA targets.

FIG. 2 is a partial view of an embodiment described herein relating to TALE-BSAb chimeric protein.

FIG. 3 is a partial view of an embodiment described herein relating to chimeric TALE-BSAb gene.

FIG. 4 is a partial view of an embodiment described herein relating to chimeric RNA.

FIG. 5 is a partial view of an embodiment described herein relating to epigenetic editor fusion proteins.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Trait variations of an organism caused by transcriptional changes of a cell or functional genomic changes, rather than sequence changes to the genome, are considered epigenetic changes and may or may not be heritable. For example, specific epigenetic marks structurally or biochemically direct gene transcription or gene silencing. For example, DNA methylation, histone modification, repressor proteins binding to silencer regions, and other transcriptional activities alter gene expression without changing the underlying DNA sequence. Thus, the transcriptional regulation allows for expression of specific genes in a particular manner, while repressing other genes. This transcriptional regulation results in variation and diversity of traits within and among organisms.

In certain instances, cell modification and cell fate can be controlled, either for initial differentiation (e.g., during the organism's development) or to reprogram a cell or cell type (e.g., during disease such as cancer, chronic inflammation, auto-immune disease, illnesses related to various microbiomes of an organism, etc.). The transcriptional factors that are involved in both cell differentiation as well as reprogramming overlap. Histone modifications play a structural and biochemical role in gene transcription, in one avenue by formation or disruption of the nucleosome structure that binds to the histone and prevents gene transcription. In addition, histone modifications have been associated with at least one of nervous system diseases (e.g., epilepsy, autism, schizophrenia, depression, mood disorder, etc.), carcinogenesis, and regenerative medicine.

As an example, inflammation associated with Crohn's disease has been traced to specific microbial gene expression of the microbiome of the gut. In fact, onset and severity in some cases can be based on the types of microflora present in the guts of the subjects. Likewise, certain gut flora that blocks the activity of certain DNA repair proteins increases the likelihood of colon cancer. It is known that the microflora of the gut can be changed rapidly and reproducibly in response to dietary changes, and the gene expression of the microflora influences the biological cells of the subject that is hosting the microflora. Thus, microbial populations that express specific genes of their own directly or indirectly influence gene expression of the biological cells of the subject that hosts the microbial populations. This occurs in plants, as well as animals and fungi.

As another example, systemic lupus erythematosus (SLE) has been shown to be related to epigenetic T cell DNA methylation, which triggers flares in those genetically predisposed to the disease. As with other autoimmune and chronic inflammatory diseases, epigenetic modifications that regulate gene expression that influence the lupus flares. In fact, there is a measurable difference between animals in germ-free environments versus those raised conventionally with regard to the onset and severity of experimental lupus. See for example, Vieira, et al. Lupus, vol. 23, no. 6, pp. 518-526 (2014), Abstract, which is incorporated herein by reference.

In addition to transcriptional modifications during development, epigenetic editing occurs during DNA repair. For example, epigenetic silencing of genes by DNA methylation or histone modifications occurs during DNA repair.

For example, the enzyme Parp1 (poly (ADP)-ribose polymerase) and its product poly (ADP)-ribose (PAR) are present at sites of DNA damage during the repair process, which recruits ALC1 for nucleosome remodeling. This in turn, causes silencing of MLH1, a DNA repair gene.

Molecular dynamics of molecular mechanisms of transcriptional events can be simulated by computer programs. For example, computer modeling of the interactions of the various transcriptional components can be conducted.

DNA methylation and chromatin remodeling are two main mechanisms for regulating gene transcription. DNA and histone proteins form a complex of chromatin, and when the physical structure of the DNA wrapped around histones is altered, gene expression is likewise altered. Furthermore, the physical structure of the chromatin can be inherited by daughter cells, allowing for the shape of the histones to be used as templates and producing a lineage-specific gene transcription program.

DNA methylation occurs primarily at CpG sites (shorthand for “—C-phosphate-G-” or “cytosine-phosphate-guanine” Highly methylated areas of DNA tend to be less transcriptionally active than lesser methylated sites. Many mammalian genes have promoter regions near or including CpG islands (regions with a high frequency of CpG sites).

In particular, the unstructured N-termini of histones are usually highly modified by at least one of acetylation, methylation, ubiquitylation, phosphorylation, sumoylation, ribosylation, or citrullination. For example, acetylation of K14 and K9 lysines of histone H3 by histone acetyltransferase enzymes is generally linked to transcriptional competence in humans. Lysine acetylation is able to directly or indirectly create binding sites for chromatin-modifying enzymes that regulate transcriptional activation. For example, histone acetyltransferases (HATs) utilize acetyl CoA as a cofactor and catalyse the transfer of an acetyl group to the epsilon amino group of the lysine side chains. This neutralizes the lysine's positive charge and weakens the interactions between histones and DNA, thus opening the chromosomes for transcription factors to bind and initiate transcription.

Likewise, histone methylation of lysine 9 of histone H3 has been associated with heterochromatin, or transcriptionally silent chromatin. Particular DNA methylation patterns are established and modified by at least three independent DNA methyltransferases, including DNMT1, DNMT3A, and DNMT3B.

In addition, various histone modifications may occur at the same time or in sequence to regulate gene transcription in a systematic or reproducible way. Likewise, histones (particularly H3 and H4 in humans) can be demethylated by the enzyme histone lysine demethylase (KDM) or other demethylases.

Certain human disorders have been associated with epigenetic alterations, including Beckwith-Wiedemann syndrome, Angleman syndrome, Prader-Willi syndrome, cancer, diabetes, obesity, DNA repair-deficiency disorder, and others. Further, epigenetic changes of biological cells have been associated with interaction with various microorganisms present as commensal organisms, or as disease-specific organisms, depending on the type of microorganism. Specifically, epigenetic alterations in cancer has led to the development of histone deacetylase (HDAC) or histone acetylase (HAT) inhibitors, as well as targeting KMT or protein arginine methyltransferases (PRMT).

In an embodiment, the subject includes a plant, animal, or fungus. In addition to the subject's own biological cells, the microorganisms that live on or in the subject may be epigenetically influenced, which has direct or indirect cross-talk with the subject's own biological cells. For example, bacteria frequently use DNA methylation for epigenetic regulation of DNA-protein interactions. Many strains of bacteria use DNA adenine methylation (rather than DNA cytosine methylation as in humans) for epigenetic regulation, and can play a role in the bacteria's infectivity (e.g., Escherichia coli, Salmonella, Vibrio, Yersinia, Haemophilus, Brucella, etc.). In certain bacterial strains, adenine methylation regulates cell cycle, gene transcription, DNA replication, mismatch repair, chromosome segregation, packaging of bacteriophage, transposase activity, or regulation of gene expression. In certain strains of fungi, cytosine methylation has been associated with inhibition of gene expression. In an embodiment, the compositions described herein can be utilized in vitro. In an embodiment, the compositions described herein can be utilized in vivo. In an embodiment, the compositions described herein can be utilized in vitro and transferred to a subject in vivo. For example, the compositions described herein can be utilized in vitro with cell lines for production of recombinant proteins and other therapeutics, or as cells in preparation for adoptive cell therapy.

In an embodiment, the composition described herein is the product of at least one vector (e.g., viral vector, plasmid, etc.). In an embodiment, the composition described herein is produced by way of integration of a coding sequence into the genome of a cell (e.g., stem cell, etc.).

In addition to the fusion protein construct, the compositions described herein can include various carriers or additives, including for example, water, acetic acid, polyvinyl alcohol, polyvinylpyrrolidone, pharmaceutically acceptable organic solvents, carboxyvinyl polymer, carboxymethylcellulose sodium, saline, glucose, sucrose, sodium polyacrylate, sodium alginate, carboxymethyl starch sodium, pectin, xanthan gum, gum Arabic, casein, agar, methylcellulose, ethylcellulose, polyethylene glycol, diglycerine, glycerine, propylene glycol, petrolatum, paraffin, stearyl alcohol, stearic acid, mannitol, lactose, sorbitol, albumin, surfactants, or other pharmaceutically acceptable carriers or additives. Likewise, in an embodiment, a kit for use or distribution of the compositions described herein is also included. In an embodiment, the kit further includes any accessories, packaging, or pharmaceutically acceptable carriers or additives as described herein.

The compositions described herein can be formulated and administered in a manner consistent with the application. In an embodiment, the dosage form of administration includes at least one of injection, oral administration, parenteral administration, topical administration, transplantation or implantation of one or more cells, or other administration. In an embodiment, the composition described herein is administered in a form including at least one of a tablet, capsule, granule, powder, solution, syrup, spray, liniment, fluid drop, cream, lotion, or ointment.

Single molecule real-time sequencing can be utilized to directly detect epigenetic marks in microorganisms by measuring methylation or other modifications as the DNA molecule is being sequenced.

In an embodiment disclosed herein, it is desirable to edit epigenetic marks in a site-specific manner. In this regard, in an embodiment, a fusion protein is described that is capable of site-specific targeting or targeting to a conserved domain common to a target nucleic acid, and includes at least one DNA binding element (e.g., DNA binding agent including a transcription factor, PNA hybridization agent, zinc finger, guide RNA for CRISPR/Cas, etc.) joined (e.g. covalently) to a flexible linker, and the flexible linker joined to at least one single chain variable fragment (scFv) lacking disulfide bonds and with a specific binding region complementary to at least one endogenous epigenetic editing agent. In an embodiment, the lack of disulfide bonds is required for the fusion protein to act intracellular. In an embodiment, the flexible linker allows for structural negotiation of the fusion protein with the nucleic acid target site.

In an embodiment, the fusion protein includes a DNA binding element that includes a transcription factor joined to a flexible linker and the flexible linker joined to at least one effector binding element, such as a single chain variable fragment lacking disulfide bonds, or an aptamer, specific for at least one endogenous epigenetic editing agent. The effector binding element binds to the endogenous epigenetic editing agent, preferably at a non-functional site as described herein.

For example, in an embodiment, the epigenetic effector binding element binds to a non-functional site of the endogenous epigenetic editing agent (thus avoiding any interference with the function of the epigenetic editing agent). For example, the Spt-Ada-Gcn5 Acetyltransferase (SAGA) complex is a highly conserved histone acetyltransferase complex in yeast that activates transcription through acetylation and deubiquitination of nucleosomal histones. In the SAGA complex, the chromatin-binding domains are clustered in one highly flexible face of the complex. Thus, in an embodiment, SAGA is at least one of the endogenous epigenetic editing agents and the effector binding element binds to a site other than the chromatin-binding domain face of the complex, thus preserving its function.

In an embodiment, the cell includes a biological cell of the subject (e.g., muscle cell, bone cell, fat cell, neuron, skin cell, blood cell (e.g., can include red blood cell, white blood cell, red blood cell ghost, platelet, etc.), epithelial cell, connective tissue cell, any precursor cells thereof, or any stem cells thereof, including embryonic stem cells, an embryonic stem cell line, immortalized cell line, pluripotent stem cells, or totipotent stem cells). In an embodiment, the biological cell includes at least one of a diseased cell (e.g., cancer cell, infected cell, etc.). In an embodiment, the diseased cell includes infected cells such as cells infected with at least one virus, bacteria, fungus, mycoplasma, etc. In an embodiment, the biological cell includes at least one cell with abnormal genetic material (e.g., can include one or more abnormal chromosome, extra or missing chromosome(s) or portions thereof, one or more mutations in one or more genes, etc.).

Various embodiments described herein are applicable to a number of animals, including but not limited to domesticated or wild agricultural animals, companion animals, rodents or vermin, or other domesticated or wild animals including but not limited to cow, horse, goat, sheep, goat, llama, alpaca, pig, hog, boar, bison, yak, buffalo, worm, chicken, turkey, goose, duck, fish, crab, lobster, oyster, shrimp, mussels, other shell fish, donkey, camel, mule, oxen, dog, cat, mouse, rat, hamster, rabbit, chinchilla, guinea pig, gerbil, ferret, elephant, bear, tiger, lion, dolphin, alligator, crocodile, whale, frog, toad, lizard, gecko, chameleon, raccoon, cougar, mountain lion, monkey, chimpanzee, gorilla, orangutan, ape, baboon, or other primate, giraffe, pigeon, pheasant, grouse, zebra, ostrich, bullock, water buffalo, carabao, snake, reindeer, caribou, elk, insect, spider, antelope, deer, moose, pony, chiliquene, cormorant, parrot, parakeet, etc. or any hybrid thereof. In an embodiment, one or more gametes are modified such that hybrids, including cross-species hybrids, are generated from the fertilization. In an embodiment, the animal includes one or more reptile, amphibian, mammal, fish, or bird.

In an embodiment, the biological cell includes a plant cell. In an embodiment, the plant cell includes a plant stem cell. In an embodiment, the plant stem cell includes a cell isolated from the meristem of a plant (e.g., the apical meristem or lateral meristem). In an embodiment, the plant stem cells are isolated from meristematic tissues such as the root apical meristem, shoot apical meristem, or vascular system ((pro) cambium or vascular meristem, for example). In an embodiment, plant stem cells are isolated from cambium.

Various embodiments described herein are applicable to a number of plants, including but not limited to grass, fruit, vegetable, flowering trees and plants (e.g., ornamental plants, fruit plants, such as apple and cherry, etc.), grain crops (e.g., corn, soybean, alfalfa, wheat, rye, oats, barley, etc.), other food or fiber crops (e.g., canola, cotton, rice, peanut, coffee, bananas, sugar cane, melon, cucumber, sugar beet, quinoa, cassava, potato, onion, tomato, strawberry, cannabis, tobacco, etc.), or other plants (including but not limited to banana, bean, broccoli, castorbean, citrus, clover, coconut, Douglas fir, Eucalyptus, Loblolly pine, linseed, olive, palm, pea, pepper, poplar, truf, Arabidopsis thaliana, Radiata pine, rapeseed, sorghum, or Southern pine. Most of the calories consumed by humans come from members of the grass family (e.g., wheat, corn [maize], rice, oats, barley, sorghum, millet, rye, etc.), and grasses make up at least a quarter of all vegetation on Earth, rendering these important food crops worldwide. Various embodiments described herein are applicable to plant cells, seeds, pollen, fruit, zygotes, etc., as disclosed.

In an embodiment, the one or more modifications of biological cells described herein are reversible. In an embodiment, the one or more modifications occur in the biological cell nucleus. In an embodiment, the one or more modifications occur in the mitochondria of the biological cells.

In an embodiment, the construct delivered to a biological cell (that can be located, for example, in vitro, in vivo, in utero, ex vivo, etc.) includes a nucleic acid construct (e.g. encoded as part of a vector), a protein construct, or a combination thereof. In an embodiment, a polycistronic vector is utilized (e.g., bicistronic, tricistronic, etc.) for efficiency. In an embodiment, multiple vectors are utilized to generate the components of the construct utilized. In an embodiment, the construct is generated ex vivo and delivered to a biological cell. As described herein, in an embodiment, the biological cell is located in a subject.

In an embodiment, the vector includes a promoter operatively linked to one or more nucleic acids desired for transcription and optionally translation for use as a construct in various embodiments described herein. In an embodiment, the promoter of at least one of the vectors utilized to generate the construct described herein is responsive to an exogenous activator or inducer. Thus, in an embodiment, delivery of the inducer causes expression of the nucleic acid(s) of the vector. In an embodiment, expression of the target gene(s) results in generation of mRNA from the nucleic acid(s) of the vector. In an embodiment, a chimeric nucleic acid/fusion protein is constructed as described herein. In an embodiment, a chimeric nucleic acid/fusion protein is generated by ligating various components together based on restriction fragments. In an embodiment, the chimeric nucleic acid/fusion protein is administered to the biological cell subsequent to assembling the construct. For example, a guide RNA is utilized with a protein construct wherein the guide RNA includes a DNA binding element and the protein construct includes an epigenetic effector binding domain, and these two components are joined by a linker (e.g., an aptamer). Thus, in an embodiment, the chimeric nucleic acid/protein construct does not require transcription for at least one nucleic acid component but is instead utilized as “raw message,” whereas the protein component is transcribed and translated prior to use. In an embodiment, the techniques for transcription and/or translation are conducted prior to delivering to the biological cell. In an embodiment, the nucleic acid is introduced in to the biological cell by way of injection, electroporation, etc. of “raw message” not contained in a vector. In an embodiment, the nucleic acid is contained within a vector, but it is not transcribed. In an embodiment, the effector binding domain is a bi-specific or multi-specific antibody.

In an embodiment, the vector is a non-viral vector and includes, for example, a plasmid, an episome, lipoplex, liposome, minichromosome, native RNA, modified RNA, native DNA, or modified DNA. In an embodiment, the vector is a viral vector and includes, for example, lentiviral vector, pox viral vector, alphaviral vector, herpes viral vector, adenoviral vector, adeno-associated viral vector, retroviral vector, vaccinia viral vector, or other viral vector. In an embodiment, the liposome is a bubble liposome (e.g., that is configured to be controlled by ultrasound).

In an embodiment, the nucleic acid target site includes at least a portion of a promoter or enhancer site (e.g., H3K4mel (methylated) is associated with an enhancer region, while H3K4me3 (methylated) is associated with a promoter region). In turn, H3K27ac (acetylated) and H3K9ac (acetylated) are increased with the activation of enhancer and promoter regions. Simultaneous histone modifications (e.g., methylation and acetylation) are generally more effective than either by itself. Additionally, targeting specific short genomic regions associated with super-enhancer sites can alter enhancers from being transcriptionally active to inactive or pausing transcription, or resume transcription if transcriptional activation is desired.

In an embodiment, a nucleic acid target site includes at least a portion of an oncogene, a tumor suppressor gene, a gene involved in cell differentiation, or a neuronal regulation gene. In an embodiment, a nucleic acid target site includes at least a portion of nucleic acid upstream from a promoter or enhancer site.

In an embodiment, binding of the DNA binding element to a nucleic acid target site results in the transcription of a specific target gene enhancer. Thus, in an embodiment, gene transcription of at least one specific target gene is indirectly initiated by the DNA binding element described herein binding to the transcription start site (promoter region) of enhancer sequences that then operate to initiate transcription of the specific target gene. Many enhancers (and their start sites) of specific genes have been identified, and additional enhancers may be elucidated by genome-scale 5′RACE (Rapid Amplification of cDNA Ends) or CAGE (Cap Analysis of Gene Expression), which detect transcriptional start sites. Super-enhancers are a subset of enhancers that are associated with genes related to cell identity and genetic risk of disease. Thus, in an embodiment, the DNA binding element binds at or near a super-enhancer transcription start site.

Non-limiting examples of neuronal regulation genes include SHC3 gene, NMDA receptor genes (e.g., NR1A or NR2C genes), or dopamine receptor genes (e.g., DRD1 or DRD2). Non-limiting examples of oncogenes include ras gene, MYCN gene, c-Myc gene, aurora kinase gene, cytoplasmic tyrosine kinase (e.g., BTK genes, SYk-ZAP70 genes, Src-family kinase genes), other tyrosine kinase receptor genes (e.g., EGFR gene, PDGFR gene, VEGFR gene), intracellular serine or threonine kinase or subunits (e.g., RAF1 gene or aurora kinase gene), transcription factor genes (e.g., MYCN gene or c-Myc gene), signal transduction adapter proteins (e.g., GRB2 gene or SHC gene), or genes for regulating GTPase activity (e.g., ras gene). Non-limiting examples of tumor suppressor genes include p53 gene, PTEN gene, p57KIP2 gene, PTC gene, TSC1 gene, TSC2 gene, EXT1 gene, EXT2 gene, p16INK4a gene, p21 gene, APC gene, RASSF1 gene, RB gene, NF1 gene, NF2 gene, p73 gene, DPC4 gene, WT1 gene, VHL gene, p19 gene, MSH2gene, MLH1 gene, BRCA1 gene, BRCA2 gene, CHEK2gene, PMS2 gene, DCC gene, or Maspin gene. Non-limiting examples of genes associated with cell differentiation or cell maintenance include OCT 3 and 4 genes, NANOG gene, KLF4 gene, MYC gene, p16INK4a gene, or MYCN gene.

Thus, histone acetylation is related to DNA demethylation and relaxing nucleosomes, allowing transcription factors to bind to DNA and initiate gene transcription. Likewise, histone deacetylation results in condensation of nucleosomes and DNA methylation, which pauses gene transcription. Histone phosphorylation and dephosphorylation occurs by way of many different enzymes (kinases for phosphorylation, phosphatases for dephosphorylation, etc.) and can regulate cell cycle as well as transcription of genes related to cell cycle, apoptosis, and others. Methylation of histones has been linked to activation of gene transcription and silencing of gene loci, depending on both the location and number of methylation sites. Histone ubiquination is associated with regulating histone methylation and may also play a role in direct regulation of gene transcription. Likewise, histone sumoylation and proline isomerization each occur at several of the same lysine residues that are targets for acetylation or ubiquitination and may compete with the same in order to regulate gene expression. Ubiquitination is regulated by ubiquitin-activating enzymes, ubiquitin-conjugating enzymes, or ubiquitin transferases (ubiquitin ligases), and deubiquitination is regulated by deubiquination enzymes (DUBs).

By contrast, deamination converts arginine residues to citrulline residues, which thereby inhibits methylation of those arginine residues and inhibits gene transcription. ADP ribosylation occurs at DNA duplex damage, and is regulated by a pair of enzymes MART (mono(ADP-ribosyl)transferase) and PART (poly (ADP-ribosyl) transferase).

In an embodiment, sumoylation i.e., addition of SUMO (small ubiquitin-like modifier) proteins, is utilized. This is a rather potent negative regulator. In addition, other modifications can be utilized, such as palmitoylation, isomerization, or ADP-ribosylation.

In an embodiment, the flexible linker includes at least one amino acid of glycine, serine, phenylalanine, glutamine, or threonine. In an embodiment, the flexible linker includes at least three repetitions of [(glycine-glycine-glycine-glycine)-serine]3 or [glycine-glycine-glycine-glycine-serine-glutamine-phenylalanine-glycine-serine-glycine-glycine]. In an embodiment, the flexible linker is approximately 5 amino acids in length, approximately 10 amino acids in length, approximately 15 amino acids in length, approximately 20 amino acids in length, approximately 25 amino acids in length, or any length therebetween. In an embodiment, the at least one DNA binding element has a specific region (e.g., DNA binding domain) complementary to a nucleic acid target site. In an embodiment, the nucleic acid target site includes a targeted region of at least one promoter region, a genetic super-enhancer region, or adjacent DNA sequences.

In an embodiment, the fusion protein described herein is nimble and therefore highly effective at finding and binding to the specifically directed nucleic acid target site. In an embodiment, the fusion protein described herein is highly selective for activating gene transcription by specifically bridging at least one endogenous epigenetic effector agent with a nucleic acid target site, thus effectively tethering the epigenetic effector agent to the nucleic acid target site. In an embodiment, the fusion protein is long enough to reach the nucleosomal histone(s) associated with the nucleic acid target site. See, for example, the Figures herein.

In an embodiment, a disabled Cas9 molecule coupled to a chimeric nucleic acid (e.g., RNA) and joined to an epigenetic effector agent. CRISPR (Clustered Regularly Interspaced Short Palindromic Repeats) utilizes the nuclease Cas9 to induce double stranded DNA breaks that are targeted to specific locations in the genome through the use of synthetic RNA (guide RNA) that directs Cas9. In an embodiment, the CRISPR system is utilized as described in embodiments herein.

In an embodiment, a DNA binding element is a transcription factor including a helix-turn-helix transcription factor, a leucine zipper transcription factor, winged helix-turn-helix transcription factor, a zinc finger transcription factor or a pioneer transcription factor. In an embodiment, the pioneer transcription factor includes at least one of FOXA, Groucho TEL, Gal4, Zld, VPOU, Oct3/4, SoxB1, KLF4, Ascl1, Pax7, PU.1, GATA4, GATA1, CLOCK, p53, or homologues thereof. In an embodiment, the DNA binding element includes at least one TALE protein (transcription activator-like effector). For example, TALEs are DNA binding proteins that contain a repeated highly conserved region, thus providing a nucleic acid binding site.

In an embodiment, a zinc finger protein includes a Cys2-His2 zinc finger protein. Specifically, in an embodiment, a zinc finger protein that includes a highly conserved region of approximately 75 amino acids at the NH2 terminus named Kruppel-associated box (KRAB), which includes subdomain A and subdomain B. KRAB containing zinc finger proteins are known to bind specific endogenous retro-elements (EREs) and regulate gene expression.

In an embodiment, a nucleic acid target site includes at least one oncogene, tumor suppressor gene, or other disease-related gene.

In an embodiment, the DNA binding element includes at least one of DNA, RNA, LNA (locked nucleic acid), or PNA (peptide nucleic acid). LNA or locked nucleic acid is an inaccessible RNA molecule that has been modified at the ribose moiety such that an extra bridge from the 2′oxygen and 4′carbon that “locks” the ribose in the 3′-endo (North) conformation. LNAs are capable of detecting short RNA and DNA targets, as well as being highly stable due to the resistance to exonucleases or endonucleases. PNA or peptide nucleic acid is an artificially synthesized polymer that has a repeating N-(2-aminoethyl)-glycine backbone linked by amide bonds, whereas DNA and RNA have a deoxyribose and ribose sugar backbone, respectively.

In an embodiment, the epigenetic editing agent endogenously functions to add or remove epigenetic marks from the endogenous histone. In an embodiment, the epigenetic marks include one or more of a methyl group, acetyl group, phosphate, or ubiquitin. In an embodiment, the epigenetic editing agent is configured for a specific level of modification (e.g., methylation, acetylation, etc.) such that 1, 2, 3, etc. modifications are added. In an embodiment, the epigenetic editing agent is configured to modify at least one histone by at last one of acetylation, phosphorylation, methylation, ubiquitination, sumoylation, proline isomerization, deamination, biotinylation, O-GlcNAcylation (O-linked N-acetylglucosaminylation), or ADP ribosylation in a site-specific manner. In an embodiment, the fusion protein described herein inhibits or activates at least one histone chaperone that acts on a nucleosome. For example, histone chaperones include TFIID, NAP1, and TAFI beta, as well as others.

In an embodiment, the epigenetic editing agent is an enzyme. In an embodiment, the at least one epigenetic editing agent includes at least one of histone deacetylase, histone deacetylase, histone methyl transferase, histone demethylase, DNA methyl transferase, DNA demethylase, DNA ligase, other ligases, ubiquitinase, ubiquitin ligase, phosphatase, or a phosphokinase.

In an embodiment the DNA binding element is a pioneer transcription factor that includes at least one of FOXA, Groucho TEL, Gal4, Zld, VPOU, Oct3/4, SoxB1, KLF4, Ascl1, Pax7, PU.1, GATA4, GATA1, CLOCK, p53, or homologues thereof.

For example, in an embodiment, a pioneer transcription factor is structurally and functionally configured to bind specifically to a common regulatory element and relax heterochromatin such that other transcription factors related to DNA transcription are able to access the chromatin. For example, transcription factors that have the highest reprogramming activity are able to engage the nucleic acid target sites on nucleosomal DNA, which is typically “closed” to transcription and is associated with non-transcriptionally active (or “silenced”) genes. The “closed” chromatin also shields the embedded nucleic acids from nuclease probes, such as DNAse I, thus a particular stretch of a chromosome can be tested as to whether it is transcriptionally “closed,” for example by electrophoretic mobility shift assays, DNase I footprinting, sequential transcription factor and core histone ChIP (chromatin immunoprecipitation), and others. However, heterochromatin (which is considered a “higher order chromatin structure) is resistant to even pioneer factor binding. Thus, there are various stages of chromatin structure that are more or less accessible to gene transcription. Generally, the “closed” chromatin lacks hypersensitivity to nuclease enzymes (e.g., DNAse) and lacks a consistent histone modification pattern.

For example, binding of pioneer factors establishes transcriptional competence of the chromatin as it allows for the binding of other transcription factors, cofactors, and enzymes. Thus, in an embodiment, the compositions described herein include a DNA binding element that is a pioneer factor, as described, and configured to bind the nucleic acid target site of closed (nuclease resistant) chromatin. In an embodiment, the compositions herein that include a pioneer factor are capable of modifying “closed” chromatin to “open” chromatin, and allowing for activation of transcription by allowing or recruiting additional factors to bind. In an embodiment, the compositions described herein include a DNA binding element that is a secondary factor that binds in conjunction with or subsequent to one or more pioneer factors, and operates to initiate gene transcription.

For example, Ascl1 is a pioneer factor that binds “closed” chromatin and recruits Brn2 and Ascl1 for differentiation of inducible neurons into functional glutaminergic neurons.

Likewise, hepatocyte-like cells induced from fibroblasts by the initial binding of the pioneer factor FOXA, which allows for further binding of HNF4a, HNF1a, and/or GATA 4.

For example, pioneer factors utilize nucleosome occupancy at gene enhancer sites as a functional binding target, and are ATP-independent. Nucleosome occupancy can be determined by testing several associated factors, including analysis of DNA sequences, transcription factors, chromatin remodeling enzymes, and transcriptional machinery. Once bound, pioneer factors are stabilized by the cooperative binding of subsequent transcription factors, either as the transcription factors also bind DNA, or bind by protein-protein interactions, and gene transcription is initiated.

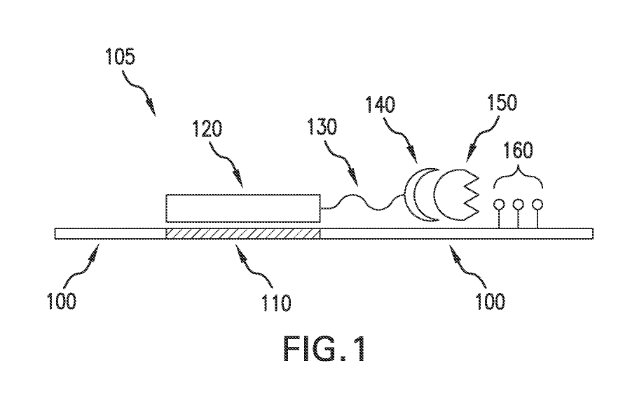

As described in FIG. 1, in an embodiment, a fusion protein 105 includes chromosomal DNA 100 in which a genomic DNA target 110 is situated, as well as epigenetic marks 160. As depicted, in an embodiment, a DNA binding element 120 is joined by a flexible linker 130 to an effector binding element 140 that is complementary to (therefore configured to specifically bind to) an epigenetic effector 150. As described herein, the genomic DNA target includes one or more of a promoter or enhancer element.

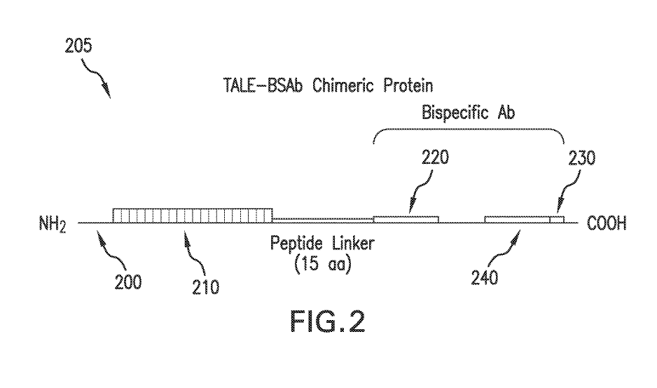

As described in FIG. 2, in an embodiment, a fusion protein 205 includes a transcription activator-like effector (TALE) protein is joined in tandem with an epigenetic effector binding protein, such as a bispecific antibody (BSAb) which captures epigenetic effector enzymes. In an embodiment, the genomic DNA 200 includes a target site for the TALE DNA Binding Protein 210, which is joined by the peptide linker (15aa) and Anti-HDAC-SCFv (Anti-Histone Deacetylase-Short Chain Variable Fragment) 220 as well as Anti-DNMT-SCFv (Anti-DNA methyl transferase-Short Chain Variable Fragment) 240 and NLS (nuclear localization sequence(s)) 230, as the protein sits from the 5 prime (NH2) to 3 prime (COOH) direction. As described herein, the linked BSAb recruits endogenous histone deacetylase (HDAC) and DNA methyl transferase (DNMT) which deacetylate histones and methylate DNA.

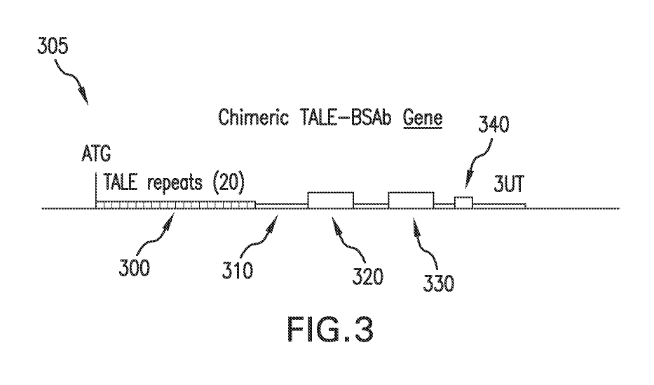

As described in FIG. 3, in an embodiment, a genetic construct 305 includes TALE (transcription activator-like effector repeats 20) 300, a peptide linker 310, HDAC-SCFV (Histone Deacetylase-Short Chain Variable Fragment) 320 and 330, and NLS (nuclear localization sequence(s)) 340, read from the 5 prime start site (ATG) to the 3 prime untranslated (UT) direction.

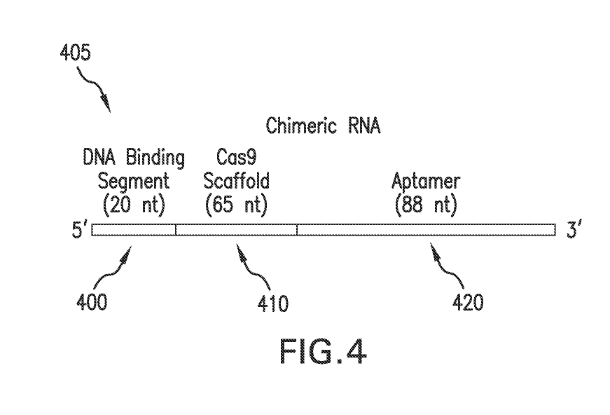

As described in FIG. 4, in an embodiment, a chimeric RNA 405 is constructed from a DNA binding segment of 20 nucleotides (20nt) 400, a Cas 9 scaffold of 65 nucleotides (65nt) 410, and an aptamer of 88 nucleotides (88nt) 420, from the 5 prime to 3 prime direction.

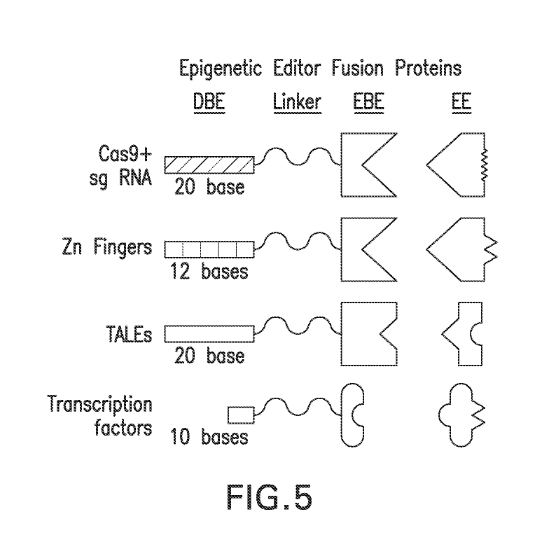

As described in FIG. 5, various epigenetic editor fusion proteins are depicted for various embodiments as described herein. For example, a Cas9+single guide RNA fusion protein includes an approximate 20 base DNA Binding Element (DBE), joined by a flexible linker to an Effector Binding Element (EBE) (e.g., single chain Fv, aptamer, etc.), which is complementary and therefore configured to specifically bind a target Epigenetic Effector (EE) (e.g., DNA methyl transferase, DNA demethylase, histone methylate, histone demethylase, histone acetylase, histone deacetylase, etc.). In another example, a Zinc Finger with an approximate 20 base DBE is joined by a flexible linker to an EBE that is complementary and thus configured to be highly specific to binding an EE. In another example, a transcription activator-like effector (TALE) protein of approximately 20 bases is joined by way of a flexible linker to an EBE wherein the EBE is complementary to an EE, and thus configured to be highly specific for binding to the EE. Likewise, in another example, a transcription factor of approximately 10 bases is joined by way of a flexible linker to an EBE wherein the EBE is complementary to an EE, and thus configured to be highly specific for binding to the EE.

### Prophetic Examples

### Prophetic Example 1

### Gene Targeting with Multiple Epigenetic Effectors Using a TALE-Bispecific Antibody (BSAb) Protein to Modify Allogeneic Cells for Adoptive Cell Therapy

Lymphocytes are epigenetically modified by targeting the human beta-2 microglobulin (β2-MG) gene with epigenetic effector enzymes. Repression of β2-MG gene expression reduces or eliminates the assembly and cell surface expression of Class I HLA proteins thus avoiding an alloimmune response. A genomic DNA binding element, for example, a transcription activator-like effector (TALE) protein is expressed in tandem with an epigenetic effector binding protein, such as a bispecific antibody (BSAb) which captures epigenetic effector enzymes (See FIGS. 1 and 2). The TALE protein binds genomic DNA proximal to the promoter enhancer region of the β2-MG gene and the linked BSAb recruits endogenous histone deacetylase (HDAC) and DNA methyl transferase (DNMT) which deacetylate histones and methylate DNA, respectively, to repress transcription of the β2-MG gene.

A DNA binding element which binds to the promoter-enhancer region of the beta 2-microglobulin (β2-MG) gene is derived from a TALE protein. DNA target sequences, including promoter-enhancer elements and transcription factor binding sites for the β2-MG gene can be adapted for use with various embodiments, such as this one. (See e.g., the website: GeneCards® at worldwide web:.genecards.org/cgi-bin/carddisp.pl?gene=B2M), and methods to design TALE proteins that bind to selected DNA sequences can be adapted for use with various embodiments, including this one (see e.g., Moscou et al., Science 326: 1501, 2009 and U.S. Patent Application Publication No. 2015/0056177, each of which is incorporated herein by reference).

For example, chimeric TALE proteins that specifically recognize selected DNA targets approximately 20 nucleotides in length are known (see e.g., U.S. Patent Application Publication No. 2015/0056177, Ibid.).

A chimeric protein is designed with a TALE DNA binding protein; a peptide linker and a BSAb which binds two epigenetic effector enzymes. See FIG. 2. A flexible peptide linker connects the TALE protein to a BSAb which recruits epigenetic enzymes to the genomic DNA target site, i.e., the β2-MG promoter-enhancer site. The flexible peptide linker is a repeated sequence, for example, a triple repeat: [glycine-glycine-glycine-glycine-serine]₃ which connects to the amino-terminus of a BSAb. The BSAb is comprised of two single chain variable region fragments (SCFvs) joined in tandem which recognize and specifically bind to HDAC2 and DNMT1. SCFvs which bind to each enzyme may be obtained by screening phage display libraries comprised of SCFv (see e.g., Kruif et al., Proc. Natl. Acad. Sci. USA 92: 3938-3942, 1995 and Rader et al., Current Opinion Biotechnology 8: 503-508, 1997; each of which is incorporated herein by reference). For example, bacteriophage expressing a library of approximately 3×10⁸ SCFv may be screened for binding to HDAC2, a histone deacetylase that removes acetyl groups from nucleosome core histones leading to repressed gene expression (see e.g., Delcuve et al., Clinical Epigenetics 4:5, 2012, which is incorporated by reference herein). SCFv which bind but do not inhibit the function of HDAC2 are identified using enzyme assays in vitro.

Recombinant HDAC 2 protein and HDAC assays are able to be adapted, for example, from: Reaction Biology Corp., Malvern, Pa. A SCFv specific for DNMT1 but devoid of function-inhibitory activity is obtained by screening and enzymatic assay (DMNT1 protein and assays are available from Reaction Biology Corp., Malvern, Pa.). Methods and peptide sequences to construct BSAb from SCFvs are known (see e.g., Mack et al., Proc. Natl. Acad. Sci. USA 92: 7021-7025, 1995, which is incorporated herein by reference). Moreover functional antibody fragments without disulfides which are stable in the cell cytoplasm and nucleus are constructed (see e.g., Seo et al., Protein Science 18: 259-267, 2008, which is incorporated herein by reference). Finally a nuclear localization sequence (see e.g., Dingwall et al., J. Cell Biol. 107: 841-849, 1988, which is incorporated herein by reference) is fused to the carboxy terminal end of the chimeric protein.

The chimeric TALE-linker-BSAb protein is encoded in a lentiviral expression vector for transduction of lymphocytes and expression of the epigenetic modifier. The gene (see FIG. 3) encoding the chimeric TALE-BSAb protein is transferred to a lentiviral vector (see e.g., U.S. Pat. No. 7,939,059, which is incorporated herein by reference). Infection of peripheral blood lymphocytes with the recombinant lentivirus results in integration of the vector sequences at random sites (i.e., not targeted) in the genomic DNA of the lymphocytes and production of the chimeric epigenetic modifier protein. (Protocols and lentiviral expression vectors are able to be adapted from Invitrogen Corp., Carlsbad, Calif.; see e.g., User Manual: “ViraPower™ HiPerform™ Lentiviral Expression Systems,” which is incorporated herein by reference).

For example, to infect a flask of lymphocytes, an aliquot of a titered recombinant lentivirus stock is diluted in fresh media (e.g., RPMI1640 with 10% fetal bovine serum available from Sigma-Aldrich, St. Louis, Mo.) so as to yield a multiplicity of infection of approximately 1.0 transducing units per cell. The lymphocytes are centrifuged briefly and the spent culture media is replaced with the aliquot of diluted recombinant lentivirus. The cells and lentivirus are incubated overnight in a tissue culture flask at 37° C. in 5% CO2; then, on the following day the lentivirus containing media is replaced by fresh media and incubated overnight. On the third day the cells are placed in selective media (e.g., media containing blasticidin) to select for stably transduced cells containing the lentiviral vector. After in vitro culture for approximately 7 days lymphocytes resistant to blasticidin are sorted using a fluorescence activated cell sorter (e.g., FACSAriaIII® available from Becton Dickinson, Franklin Lakes, N.J.). Antibodies recognizing pan HLA class I (e.g., L243 antibody from Sigma-Aldrich, St. Louis, Mo.) are used to isolate HLA-negative lymphocytes which are retained for adoptive immunotherapy. HLA class I-negative lymphocytes are expanded and tested periodically to monitor the repression of β2-MG expression and the lack of HLA class I on the cell surface.

### Prophetic Example 2

### Epigenetic Editing of a Breast Cancer Gene, BRCA1, Using a Chimeric RNA and Disabled CAS9 to Induce Demethylation of the Promoter Region of BRCA1

Tumor suppressor genes, such as BRCA1 are frequently hyper-methylated around their promoter region and their expression is repressed. In order to regain BRCA1 expression site-specific demethylation is induced by a chimeric RNA which binds and localizes a Ten-Eleven Translocation (TET) dioxygenase proximal to the BRCA1 promoter. For example, TET2 dioxygenase induces demethylation and activation of targeted genes (see e.g., Chen et al., Nucleic Acids Research 42: 1563-1574, 2013, which is incorporated herein by reference). A recombinant lentivirus is constructed to express the chimeric RNA molecule and disabled CAS9 (dCAS9) endonuclease, which is necessary for targeting the chimeric RNA to the BRCA1 gene. Transduction of mammary cells with the recombinant lentivirus vector and expression of dCAS9 and the chimeric RNA which binds TET2, induces demethylation of BRCA1 genes and allows expression of BRCA1 to reduce breast cancer risk.

The chimeric RNA contains a DNA binding element which is complementary to the genomic DNA target (i.e., BRCA1); a scaffold segment which interact with dCAS9 and an aptamer segment which binds an epigenetic effector element, TET2. See FIG. 4. For example, a DNA binding element of approximately 20 nucleotides is used with dCAS9 endonuclease to target genomic DNA sites (see e.g., Int. Appl. No. WO 2014/089290, which is incorporated herein by reference). DNA sequences proximal to the BRCA1 gene promoter and known methylation sites are described (see e.g., Tapia et al., Epigenetics 3: 157-163, 2008, which is incorporated herein by reference). For example, BRCA1 genomic DNA sequences between nucleotides 1543 and 1617 contain several methylation sites which may be targeted with approximately 20-nucleotide RNAs complementary to nearby sequences. A scaffolding segment, approximately 65 nucleotides in length is placed immediately downstream (i.e., 3′) of the DNA binding segment. RNA sequences of scaffolding segments compatible with dCAS9 are described (see e.g., WO 2014/08920, and U.S. Pat. No. 8,945,839; each of which is incorporated herein by reference).

An RNA aptamer which binds TET2 is encoded 3′ of the CAS9 scaffold segment. An RNA aptamer which binds TET2 protein with high specificity is selected from an RNA aptamer library. Methods to select RNA aptamers and RNA aptamer libraries are available from TriLink Biotechnologies, San Diego, Calif. (see e.g., worldwide web: trilinkbiotech.com/about/contact.asp and Germer et al., Int. J. Biochem Mol Biol 4: 27-40, 2013; each of which is incorporated herein by reference). An RNA aptamer approximately 88 nucleotides in length is selected using recombinant TET2 protein, and its RNA sequence is determined (see e.g., Germer et al., Ibid.). The corresponding DNA sequence is placed 3′ of the scaffold segment. See FIG. 4.

### Prophetic Example 3

### Epigenetic Editing to Alter Lignin Content in California Poplar Tree

The California poplar, Populus trichocarpa, is epigenetically modified to reduce expression of an important enzyme for lignin biosynthesis. The gene for caffeoyl shikimate esterase (CSE) is epigenetically repressed to reduce the amount of lignin produced by the tree, thus facilitating tree processing for the production of paper and biofuels. Epigenetic modification is done with a chimeric RNA which contains a DNA binding element at one end, a scaffold segment for CAS9 binding and an aptamer to capture an epigenetic effector enzyme at the other end. The chimeric RNA binds near transcription elements for the lignin biosynthetic enzyme, CSE, and captures a DNA methyl transferase which methylates local cytosine residues and thus represses CSE transcription. For example a DNA methyl transferase, MET1, is bound by the aptamer segment and catalyzes DNA methylation of CpNpG and CpG near the transcription start site of the CSE gene. The chimeric RNA and a disabled CAS9 (dCAS9) endonuclease are encoded in a plant viral vector which is used to transfect the tree.

The chimeric RNA contains: A DNA binding element which is complementary to the genomic DNA target (i.e., CSE), a scaffold segment which interacts with dCAS9, and an aptamer segment which binds an epigenetic effector element. The DNA sequence of the CSE gene is used to design a guide RNA to target the promoter region and transcriptional start site of the CSE gene (which can be deduced from the sequence of the CSE gene (see e.g., Vanholme et al., Science 341, 1103-1106, 2013, which is incorporated herein by reference). For example, a DNA binding element of approximately 20 nucleotides is used with disabled CAS9 (dCAS9) endonuclease to target genomic DNA sites (see e.g., Int. Appl. No. WO 2014/089290, which is incorporated herein by reference) and tools for the design of guide RNAs with specificity for plant DNA sequences are described generally and can be adapted for particular embodiments (see e.g., Bortesi et al., Biotech. Advances 33: 41-52, 2015 which is incorporated herein by reference). A scaffolding segment, approximately 65 nucleotides in length is placed immediately downstream (i.e., 3′) of the DNA binding segment. RNA sequences of scaffolding segments compatible with dCAS9 are described (see e.g., Int. Appl. No. WO 2014/08920, Ibid. and U.S. Pat. No. 8,945,839, each of which is incorporated herein by reference). An RNA aptamer segment which binds an epigenetic effector enzyme follows the scaffold segment.

An RNA aptamer which binds a non-catalytic portion of a DNA methyltransferase is selected and attached to the scaffold segment of the chimeric RNA. For example an RNA aptamer which binds the regulatory region of Metl DNA methyltransferase (see e.g., Wada, Plant Biotechnology 22: 71-80, 2005 which is incorporated herein by reference) is selected from an RNA aptamer library. General methods to select RNA aptamers and RNA aptamer libraries are available and can be adapted for use as described herein this section (see e.g., the world wide web at trilinkbiotech.com/about/contact.asp and Germer et al., Int. J. Biochem Mol Biol 4: 27-40, 2013 which is incorporated herein by reference).

The chimeric RNA, including the guide RNA segment, scaffold segment and aptamer are encoded in a binary vector that also encodes dCAS9. The plasmid vector is transferred into Argobacterium tumefaciens and then poplar cells are transformed. Transformed plants are selected for kanamycin resistance and transplanted to soil. General methods and materials to transform plants can be adapted (see e.g., Zhou et al., New Phytologist, May, 2015. DOI: 10.1111/nph.13470 which is incorporated herein by reference). Transgenic poplar trees are tested to determine their lignin content relative to nontransgenic trees. General methods to measure lignin content can be adapted for use as described herein (see e.g., U.S. Pat. No. 6,441,272, which is incorporated herein by reference).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A composition, comprising:
a fusion protein
including at least one DNA binding element joined to a flexible linker,
and the flexible linker also joined to
at least one effector binding element with a specific binding region complementary to at least one endogenous epigenetic effector agent.

2. The composition of claim 1, wherein the flexible linker includes at least one amino acid of alanine, glycine, serine, phenylalanine, glutamine, or threonine.

3.-4. (canceled)

5. The composition of claim 1, wherein the at least one DNA binding element has a specific region complementary to a nucleic acid target site.

6. The composition of claim 5, wherein the nucleic acid target site includes at least one genomic target DNA sequence.

7. The composition of claim 5, wherein the DNA binding element includes at least a portion of a CRISPR-Cas guide RNA, a zinc finger protein, or a transcription factor.

8. The composition of claim 5, wherein the nucleic acid target site includes a promoter, super enhancer or enhancer DNA sequence region.

9. The composition of claim 1, wherein the specific binding region complementary to at least one endogenous epigenetic effector agent binds to a non-functional site of the endogenous epigenetic effector agent.

10. (canceled)

11. The composition of claim 1, wherein the DNA binding element includes at least one of DNA, RNA, LNA, PNA, a CRISPR/Cas 9/guide RNA complex, a zinc finger protein, a pioneer transcription factor, or a TALE protein.

12. The composition of claim 11, wherein the pioneer transcription factor includes at least one of FOX, Groucho TEL, Gal4, Zld, VPOU, Oct3/4, SoxB1, or homologues thereof.

13.-14. (canceled)

15. The composition of claim 1, wherein the epigenetic effector agent is an enzyme.

16. The composition of claim 1, wherein the enzyme includes at least one of a DNA methyl transferase, DNA demethylase, DNA ligase, histone deacetylase, histone demethylase, histone methylase, ubiquitinase, or histone acetylase.

17. The composition of claim 1, wherein the effector binding element includes at least one of an aptamer or a single chain variable fragment lacking disulfide bonds.

18. The composition of claim 1, wherein the effector binding element includes at least one bi-specific antibody.

19. A composition, comprising:
a vector including nucleic acid sequence encoding a fusion protein,
the fusion protein including at least one DNA binding element joined to a flexible linker,
and the flexible linker also joined to
at least one effector binding element with a specific binding region complementary to at least one endogenous epigenetic effector agent.

20. The composition of claim 19, wherein the vector includes at least one of a viral vector or non-viral vector.

21. The composition of claim 20, wherein the non-viral vector includes at least one of a plasmid, an episome, liposome, lipoplex, minichromosome, native RNA, modified RNA, native DNA, or modified DNA.

22. The composition of claim 20, wherein the viral vector includes at least one of a lentiviral vector, pox viral vector, alphaviral vector, herpes viral vector, adenoviral vector, adeno-associated viral vector, retroviral vector, vaccinia viral vector, or other viral vector.

23.-24. (canceled)

25. A method of modifying a biological cell, comprising:
delivering to a biological cell,
a fusion protein including at least one DNA binding element joined to a flexible linker, and
the flexible linker also joined to at least one effector binding element with a specific binding region complementary to at least one endogenous epigenetic effector agent.

26. The method of claim 25, wherein delivering the fusion protein to the biological cell includes at least one of, transfection, membrane fusion, or osmotic shock, squeezing cells, electroporation, lipofection, or endocytosis.

27. The method of claim 26, wherein membrane fusion includes use of an exosome or cell wall fusion.

28. The method of claim 26, wherein infection includes utilizing at least one virus to transport the fusion protein into the biological cell.

29.-37. (canceled)

38. The method of claim 25, wherein the nucleic acid sequence further encodes a promoter region for the fusion protein.

39. The method of claim 38, wherein the promotor region is configured to respond to an exogenous activator or inducer molecule.

40. A method of modifying a biological cell, comprising:
delivering to a biological cell,
a vector including nucleic acid sequence encoding a fusion protein,
the fusion protein including at least one DNA binding element joined to a flexible linker, and
the flexible linker also joined to at least one effector binding element with a specific binding region complementary to at least one endogenous epigenetic effector agent.

41. The method of claim 40, wherein delivering the fusion protein to the biological cell includes at least one of transfection, membrane fusion, or osmotic shock, squeezing cells, electroporation, lipofection, or endocytosis.

42. The method of claim 40, wherein infection includes utilizing at least one virus to transport the fusion protein into the biological cell.

43.-48. (canceled)

49. The method of claim 42, further including transferring the biological cell to a biological subject.

50. The method of claim 42, further including inducing the biological cell to replicate.

51. The method of claim 50, wherein inducing the biological cell to replicate includes inducing the biological cell to replicate at least one of in vitro, ex vivo, or in vivo.

52.-62. (canceled)
